(19) United States
(12) Patent Application Publication
(10) Pub. No.: US 2020/0402607 A1
(43) Pub. Date: Dec. 24, 2020

(54) Covariant Neural Network Architecture for Determining Atomic Potentials

(71) Applicant: The University of Chicago, Chicago, IL (US)

(72) Inventor: Imre Kondor, Chicago, IL (US)

(21) Appl. No.: 16/975,962

(22) PCT Filed: Mar. 4, 2019
(86) PCT No.: PCT/US2019/020536

§ 371 Date: Aug. 26, 2020

Related U.S. Application Data
(60) Provisional application No. 62/637,934, filed on Mar. 2, 2018.

(51) Int. Cl. G16B 5/00; G16B 40/20; G06N 3/04; G06N 3/08; G06N 5/04

(52) U.S. Cl. CPC G16B 5/00; G16B 40/20; G06N 5/046; G06N 3/084; G06N 3/0481; G06N 3/0463

(57) ABSTRACT

Methods and systems for computationally simulating an N-body physical system are disclosed. A compound object X having N elementary parts E may be decomposed into J subsystems, each including one or more of the elementary parts and having a position vector rⱼ and state vector ψⱼ. A mural network having J nodes each corresponding to one of the subsystems may be constructed, the nodes including leaf nodes, a non-leaf root node, and intermediate non-leaf nodes, each being configured to compute an activation corresponding to the state of a respective subsystem. Upon receiving input data for the parts E, each node may compute ψⱼ from rⱼ and ψⱼ of its child nodes using a covariant aggregation rule representing ψⱼ as a tensor that is covariant to rotations of the rotation group SO(3). A Clebsch-Gordan transform may be applied to reduce tensor products to irreducible covariant vectors, and ψⱼ of the root node may be computed as output of the ANN.

### CROSS-REFERENCE TO RELATED APPLICATIONS

This application claims priority under 35 U.S.C. § 119(e) to U.S. Provisional Patent Application Ser. No. 62/637,934, filed on Mar. 2, 2018, which is incorporated herein in its entirety by reference.

### STATEMENT REGARDING FEDERALLY SPONSORED RESEARCH OR DEVELOPMENT

This invention was made with government support under grant number D16AP00112 awarded by the Defense Advanced Research Projects Agency. The government has certain rights in the invention.

### BACKGROUND

In principle, quantum mechanics provides a perfect description of the forces governing the behavior of atomic systems such as crystals and biological molecules. However, for systems larger than a few dozen atoms, solving the Schrodinger equation explicitly, on present day computers, is generally not a feasible proposition. Density Functional Theory (DFT), a widely used approximation in quantum chemistry, has trouble scaling to more than about a hundred atoms.

In view of such limitations, a majority of practical work in molecular dynamics typically foregoes modeling electrons explicitly, and falls back on the fundamentally classical (i.e., non-quantum) Born-Oppenheimer approximation, which treats atoms as solid balls that exert forces on nearby balls prescribed by so called (effective) atomic potentials. This approximation assumes that the potential attached to atom i is ϕᵢ(\hat{r}₁, . . . , \hat{r}ₖ), with \hat{r}ⱼ=rₚ_{ⱼ}−rᵢ, where rᵢ is the position vector of atom i and rₚⱼ is the position vector of its j'th neighbor. The total force experienced by atom i is then simply the negative gradient Fᵢ=−∇ᵣ_{ᵢ}ϕᵢ(\hat{r}₁, . . . , \hat{r}ₖ). Classically, in molecular dynamics ϕᵢ is usually given in terms of a closed form formula with a few tunable parameters. Know techniques in this area are usually characterized according to empirical potentials or empirical force fields.

While empirical potentials may be fast to evaluate, they are crude models of the quantum interactions between atoms, limiting the accuracy of molecular simulation. More recently, machine learning has been applied to molecular simulations, showing some promise to bridge the gap between the quantum and classical worlds by earning the aggregate force on each atom as a function of the positions of its neighbors from a relatively small number of DFT calculations. Since its introduction, the amount of research and development in so-called machine learned atomic potentials (MLAP) has expanded significantly, and molecular dynamics simulations based on this approach may be showing evidence of results that outperform other methods.

### SUMMARY

Much of the work in machine learning algorithms in area of, and related to, molecular simulations has been applied to the MLAP problem, from genetic algorithms, through kernel methods, to neural networks. However, the inventor has recognized that rather than the statistical details of the specific learning algorithm, a more appropriate focus for problems of this type may be the representation of the atomic environment, i.e., the choice of learning features that the algorithm is based on. This situation may arise in other areas applied machine learning as well. For example, such representational issues also play a role in computer vision and speech recognition. What makes the situation in Physics applications somewhat special is the presence of constraints and invariances that the representation must satisfy not just in an approximate, but in the exact sense. Rotation invariance provides instructive, contrasting examples. Specifically, if rotation invariance is not fully respected by an image recognition system, some objects might be less likely to be accurately detected in certain orientations than in others. In a molecular dynamics setting, however, using a potential that is not fully rotationally invariant would not just degrade accuracy, but would likely lead to entirely unphysical molecular trajectories.

Recent efforts in MLAP work have been shifting from fixed input features towards representations learned from the data itself, exemplified in particular by application of “deep” neural networks to represent atomic environments. It has been recognized that certain concepts from the mainstream neural networks research, such as convolution and equivariance, can be repurposed to this domain. This may reflect an underlying analogy between MLAP and computer vision. More particularly, in both domains two competing objectives need to be met for success:
1. The ability to capture structure in the input data at multiple different length (or size) scales, i.e., to construct a multiscale representation of the input image or the atomic environment.
2. The above-mentioned invariance property with respect to spatial transformations, including translations, rotations, and possibly scaling.

The inventor has further recognized that many of the concepts involved in learnable multiscale representations may be extended to create a neural network architecture where the individual “neurons” correspond to physical subsystems endowed with their own internal state. In the present disclosure, such neural networks are referred to as “N-body networks.” The structure and behavior of the resulting model follows a tradition of coarse graining and representation theoretic ideas in Physics, and provides a learnable and multiscale representation of the atomic environment that is fully covariant to the action of the appropriate symmetries. What is more, the scope of the underlying ideas a broader, meaning that N-body networks have potential application in modeling other types of many-body Physical systems, as well.

Further still, the inventor has recognized that the machinery of group representation theory, specifically the concept of Clebsch-Gordan decompositions, can be used to design neural networks that are covariant to the action of a compact group yet are computationally efficient. This aspect is related to the other recent areas of interest involving generalizing the notion of convolutions to graphs, manifolds, and other domains, as well as the question of generalizing the concept of equivariance (covariance) in general. Analytical techniques in these recent areas have employed generalized Fourier representations of one type or another, but to ensure equivariance the nonlinearity was always applied in the time domain. However, projecting back and forth between the time domain and the frequency domain can be a major bottleneck in terms of computation time and efficiency. In contrast, the inventor has recognized that application of the Clebsch-Gordan transform allows computation of one type of nonlinearity, namely tensor products, entirely in the Fourier domain. Accordingly, example methods and system disclosed herein provide for a significant improvement over other existing and previous analysis techniques, and provides the groundwork for efficient N-body networks for simulation and modeling of a wide variety of types of many-body Physical systems.

Thus, in one respect, example embodiments may involve a method for computationally simulating an N-body physical system, wherein the N-body physical system is represented mathematically as a compound object X having N elementary parts E={eᵢ}, i=1, . . . , N, each eᵢ representing one of the N bodies of the N-body physical system, wherein X is hierarchically decomposed into J subsystems, Pⱼ, j=1, . . . , J, each Pⱼ comprising one or more of the elementary parts of E, and wherein each Pⱼ is described by a position vector rⱼ and an internal state vector ψⱼ, the method being implemented on a computing device and comprising: constructing a hierarchical artificial neural network (ANN) having J nodes each corresponding to one of the J subsystems, the J nodes including m≥2 leaf nodes as ANN inputs, m=1 non-leaf root node as ANN output, and m≥1 intermediate non-leaf nodes, wherein each node is a neuron of the ANN and is configured to compute an activation corresponding to a different one of the internal state vectors ψⱼ, and wherein: for each leaf node, ψⱼ describes the internal state of a respective one of the Pⱼ subsystems having just a single elementary part eᵢ, for each given intermediate non-leaf node, ψⱼ describes the internal state of a respective one of the Pⱼ subsystems having 2≤k<N parts eᵢ that are each comprised in a child node of the given intermediate non-leaf node, and for the root node, ψⱼ describes the internal state of a subsystem Pⱼ having k=N elementary parts eᵢ that are each comprised in a child node of the root node; at the computing device, receiving input data to the leaf nodes specifying respective position vectors and respective internal state vectors of the N elementary parts E; for each given non-leaf node, computing ψⱼ from the position vectors and internal states of all the child nodes of the given non-leaf node according to a covariant aggregation rule that represents ψⱼ as a tensor object that is covariant to rotations of the rotation group SO(3); applying a Clebsch-Gordan transform to reduce tensor products of the state vectors of the nodes to irreducible covariant vectors; and computing ψⱼ of the root node as output of the ANN, to determine a simulation of the internal state of the N-body physical system.

In another respect, example embodiments may involve a computing device configured for computationally simulating an N-body physical system, wherein the N-body physical system is represented mathematically as a compound object X having N elementary parts E={eᵢ}, i=1, . . . , N, each eᵢ representing one of the N bodies of the N-body physical system, wherein X is hierarchically decomposed into J subsystems, Pⱼ, j=1, . . . , J, each Pⱼ comprising one or more of the elementary parts of E, and wherein each Pⱼ is described by a position vector rⱼ and an internal state vector ψⱼ, the computing device comprising: one or more processors; and memory configured to store computer-executable instructions that, when executed by the one or more processors, cause the computing device to carry out computational operations including: constructing a hierarchical artificial neural network (ANN) having J nodes each corresponding to one of the J subsystems, the J nodes including m≥2 leaf nodes as ANN inputs, m=1 non-leaf root node as ANN output, and m≥1 intermediate non-leaf nodes, wherein each node is a neuron of the ANN and is configured to compute an activation corresponding to a different one of the internal state vectors ψⱼ, and wherein: for each leaf node, ψⱼ describes the internal state of a respective one of the Pⱼ subsystems having just a single elementary part eᵢ, for each given intermediate non-leaf node, ψⱼ describes the internal state of a respective one of the Pⱼ subsystems having 2≤k<N parts eᵢ that are each comprised in a child node of the given intermediate non-leaf node, and for the root node, ψⱼ describes the internal state of a subsystem Pⱼ having k=N elementary parts eᵢ that are each comprised in a child node of the root node; receiving input data to the leaf nodes specifying respective position vectors and respective internal state vectors of the N elementary parts E; for each given non-leaf node, computing ψⱼ from the position vectors and internal states of all the child nodes of the given non-leaf node according to a covariant aggregation rule that represents ψⱼ as a tensor object that is covariant to rotations of the rotation group SO(3); applying a Clebsch-Gordan transform to reduce a tensor product of the state vectors of the nodes to irreducible covariant vectors; and computing ψⱼ of the root node as output of the ANN to determine the internal state of the N-body physical system.

In still another respect, example embodiments may involve an article of manufacture comprising a non-transitory computer readable media having computer-readable instructions stored thereon for computationally simulating an N-body physical system, wherein the N-body physical system is represented mathematically as a compound object X having N elementary parts E={eᵢ}, i=1, . . . , N, each eᵢ representing one of the N bodies of the N-body physical system, wherein X is hierarchically decomposed into J subsystems, Pⱼ, j=1, . . . , J, each Pⱼ comprising one or more of the elementary parts of E, and wherein each Pⱼ is described by a position vector rⱼ and an internal state vector ψⱼ, and wherein the instructions, when executed by one or more processors of a computing device, cause the computing device to carry out operations including: constructing a hierarchical artificial neural network (ANN) having J nodes each corresponding to one of the J subsystems, the J nodes including m≥2 leaf nodes as ANN inputs, m=1 non-leaf root node as ANN output, and m≥1 intermediate non-leaf nodes, wherein each node is a neuron of the ANN and is configured to compute an activation corresponding to a different one of the internal state vectors ψⱼ, and wherein: for each leaf node, ψⱼ describes the internal state of a respective one of the Pⱼ subsystems having just a single elementary part eᵢ, for each given intermediate non-leaf node, ψⱼ describes the internal state of a respective one of the Pⱼ subsystems having 2≤k<N parts eᵢ that are each comprised in a child node of the given intermediate non-leaf node, and for the root node, ψⱼ describes the internal state of a subsystem Pⱼ having k=N elementary parts eᵢ that are each comprised in a child node of the root node; receiving input data to the leaf nodes specifying respective position vectors and respective internal state vectors of the N elementary parts E; for each given non-leaf node, computing ψⱼ from the position vectors and internal states of all the child nodes of the given non-leaf node according to a covariant aggregation rule that represents ψⱼ as a tensor object that is covariant to rotations of the rotation group SO(3); applying a Clebsch-Gordan transform to reduce a tensor product of the state vectors of the nodes to irreducible covariant vectors; and computing ψⱼ of the root node as output of the ANN to determine the internal state of the N-body physical system.

These as well as other embodiments, aspects, advantages, and alternatives will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF DRAWINGS

FIG. 1 depicts a simplified block diagram of an example computing device, in accordance with example embodiments.

FIG. 2 is a conceptual illustration of two types of tree-like artificial neural network, one strict tree-like and the other non-strict tree-like, in accordance with example embodiments.

FIG. 3A is a conceptual illustration of an N-body system, in accordance with example embodiments.

FIG. 3B is a conceptual illustration of an N-body system showing a second level of substructure, in accordance with example embodiments.

FIG. 3C is a conceptual illustration of an N-body system showing a third level of substructure, in accordance with example embodiments.

FIG. 3D is a conceptual illustration of an N-body system showing a fourth level of substructure, in accordance with example embodiments.

FIG. 3E is a conceptual illustration of an N-body system showing a fifth level of substructure, in accordance with example embodiments.

FIG. 3F is a conceptual illustration of a decomposition of an N-body system in terms of subsystems and internal states, in accordance with example embodiments.

FIG. 4A is a conceptual illustration of compositional scheme for a compound object representing an N-body system, in accordance with example embodiments.

FIG. 4B is a conceptual illustration of compositional neural network for simulating an N-body system, in accordance with example embodiments.

FIG. 5 is a flow chart of an example method, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

### I. INTRODUCTION

Example embodiments of a covariant hierarchical neural network architecture, referred to herein as “N-body comp-nets,” are described herein in terms of molecular structure, and in particular, atomic potentials of molecular systems. The example of such molecular systems provides a convenient basis for connecting analytic concepts of N-body comp-nets to physical systems that may be illustratively conceptualized. For example, a physical hierarchy of structures and substructures of molecular constituents (e.g., atoms) may lend itself to a descriptive visualization. Similarly, the concept of rotational and/or translational invariance (or, more generally, invariance to spatial transformations) may be easily grasped at a conceptual level in terms of the ability of a neural network to learn to recognized complex systems regardless of their spatial orientations when presented to the neural network. And consideration of learning atomic and/or molecular potentials of such systems can help tie the structure of the constituents to their physics in an intuitive manner. However, the example of molecular/atomic systems and potentials is not, and should not, be viewed as limiting with respect to either the analytical framework or the applicability of N-body comp-nets.

More specifically, the challenges described above—namely the ability to recognize multiscale structure while maintaining invariance with respect to spatial transformation—may be met by the inventor's novel application of concepts of group representation theory to neural networks. The inventor's introduction of Clebsch-Gordan decompositions into hierarchically structured neural networks is one aspect of example embodiments described herein that makes N-body comp-nets broadly applicable to problems beyond the example of molecular/atomic systems and potentials. In particular, it supplies an analytical prescription for how neural networks may be constructed and/or adapted to simulate a wide range of physical systems, as well as address problems in areas such as computer vision, and computer graphics (and, more generally, point-cloud representations), among others.

In relation to physical systems described by way of example herein, neurons of an example N-body comp-net may be described as representing internal states of subsystems of a physical system being modeled. This too, however, is a convenient illustration that may be conceptually connected to the physics of molecular and/or atomic systems. Thus, in accordance with example embodiments, internal state may be a convenient computational representation of the activations of neurons of a comp-net. In other applications, the activations may be associated with other physical properties or analytical characteristics of the problem at hand. In either case (and in others), a common aspect of activations of a comp-net is the transformational properties provided by tensor representation and the Clebsch-Gordan decompositions it admits. These are aspects that enable neural networks to meet challenges that have previously vexed their operation. Practical applications of simulations of N-body comp-nets are extensive.

In relation to molecular structure and dynamics, N-body comp-nets may be used to learn, compute, and/or predict (in addition to potential energies) forces, metastable states, and transition probabilities. Applied or integrated in a context of larger structure, N-body comp-nets may be extended to areas of material design, such as tensile strength, design of new drug compounds, simulation of protein folding, design of new battery technologies and new types of photovoltaics. Other areas of applicability of N-body comp-nets may include prediction of protein-ligand interactions, protein-protein interactions, and properties of small molecules, including solubility and lipophilicity. Additional applications may also include protein structure prediction and structure refinement, protein design, DNA interactions, drug interactions, protein interactions, nucleic acid interactions, protein-lipid-nucleic acid interactions, molecule/ligand interactions, drug permeability measurements, and predicting protein folding and unfolding. As this list of examples suggests, N-body comp-nets may provide a basis for wide applicability, both in terms of the classes and/or types of specific problems tackled, and the conceptual variety of problems they can address.

### II. EXAMPLE COMPUTING DEVICES

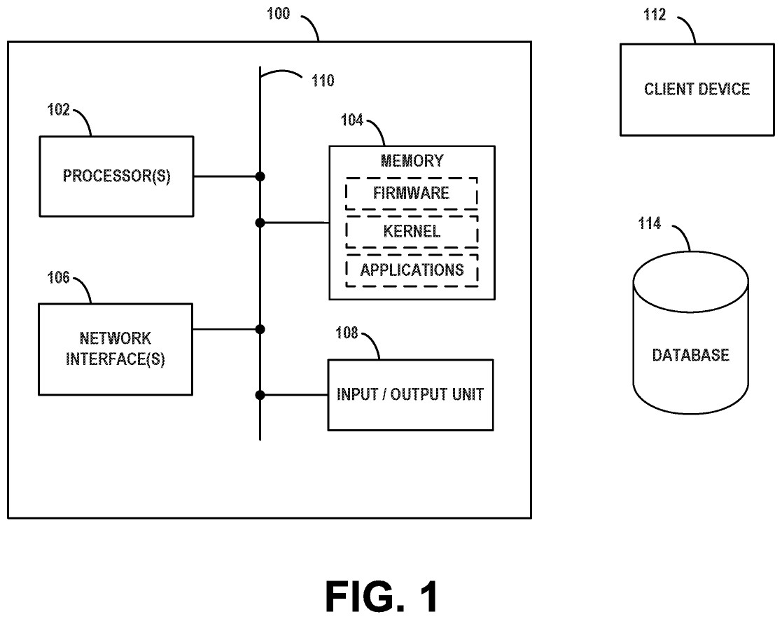

FIG. 1 is a simplified block diagram of a computing device 100, in accordance with example embodiments. As shown, the computing device 100 may include processor(s) 102, memory 104, network interface(s) 106, and an input/output unit 108. By way of example, the components are communicatively connected by a bus 110. The bus could also provide power from a power supply (not shown). In particular, computing device 100 may be configured to perform at least one function of and/or related to implementing all or portions of artificial neural networks 200, 202, and/or 400-B, machine learning system 700, and/or method 500, all of which are described below.

Memory 104 may include firmware, a kernel, and applications, among other forms and functions of memory. As described, the memory 104 may store machine-language instructions, such as programming code or non-transitory computer-readable storage media, that may be executed by the processor 102 in order to carry out operations that implement the methods, scenarios, and techniques as described herein and in accompanying documents and/or at least part of the functionality of the example devices, networks, and systems described herein. In some examples, memory 104 may be implemented using a single physical device (e.g., one magnetic or disc storage unit), while in other examples, memory 104 may be implemented using two or more physical devices. In some examples, memory 104 may include storage for one or more machine learning systems and/or one or more machine learning models as described herein.

Processors 102 may include one or more general purpose processors and/or one or more special purpose processors (e.g., digital signal processors (DSPs) or graphics processing units (GPUs). Processors 102 may be configured to execute computer-readable instructions that are contained in memory 104 and/or other instructions as described herein.

Network interface(s) 106 may provide network connectivity to the computing system 100, such as to the internet or other public and/or private networks. Networks may be used to connect the computing system 100 with one or more other computing devices, such as servers or other computing systems. In an example embodiment, multiple computing systems could be communicatively connected, and example methods could be implemented in a distributed fashion.

Client device 112 may be a user client or terminal that includes an interactive display, such as a GUI. Client device 112 maybe used for user access to programs, applications, and data of the computing device 100. For example, a GUI could be used for graphical interaction with programs and applications described herein. In some configurations, the client device 112 may itself be a computing device; in other configurations, the computing device 100 may incorporate, or be configured to operate as, a client device.

Database 114 may include input data, such as images, configurations of N-body systems, or other data used in the techniques described herein. Data could be acquired for processing and/or recognition by a neural network, including artificial neural networks 200, 202, and/or 400-B. The data could additionally or alternatively be training data, which may be input to a neural network, for training, such as determination of weighting factors applied at various layers of the neural network. Database 114 could be used for other purposes as well.

### III. EXAMPLE ARTIFICIAL NEURAL NETWORKS FOR REPRESENTING STRUCTURED OBJECTS

Example embodiments of N-body neural networks for simulation and modeling may be described in terms of some of the structures and features of “classical” feed-forward neural networks. Accordingly, a brief review of classical feed-forward networks is presented below in order to provide a context for describing an example general purpose neural architecture for representing structured objects referred to herein as “compositional networks.”

A prototypical feed-forward neural network consists of some number of neurons { [symbol] } arranged in L+1 distinct layers. Layer [symbol] =0 is referred to as the “input layer,” and is where training and testing data enter the network, while the inputs of the neurons in layers [symbol] =1, 2, . . . , L are the outputs { [symbol] } of the neurons in the previous layer. Each neuron computes its output, also called its “activation,” using a simple rule such as

[symbol]

=σ( [symbol] + [symbol] ) (1)

where the { [symbol] } weights and { [symbol] } biases are learnable parameters, while σ is a fixed nonlinearity, such a sigmoid function or a ReLU operator. The output of the network appears in layer L, also referred to as the “output layer.” As computational entities or constructs implemented as software or other machine language code executable on a computing device, such as computing device 100, neural networks are also commonly referred to as “artificial neural networks” or ANNs. The term ANN may also refer to a broader class of neural network architectures than feed-forward networks, and is used without loss of generality to refer to example embodiments of neural networks described herein.

During training of a feed-forward neural network, training data are input, and the output layer results are compared with the desired output by means of a loss function. The gradient of the loss may be back-propagated through the network to update the parameters, typically by some variant of stochastic gradient descent. During real-time or “live” operation, testing data, representing some object (e.g., a digital image) or system (e.g., a molecule) having an unknown a priori output result, are fed into the network. The result may represent a prediction by the network of the correct output result to within some prescribed statistical uncertainty, for example. The accuracy of the prediction may depend on the appropriateness of the network configuration for solving the problem, as well as the amount and/or quality of the training.

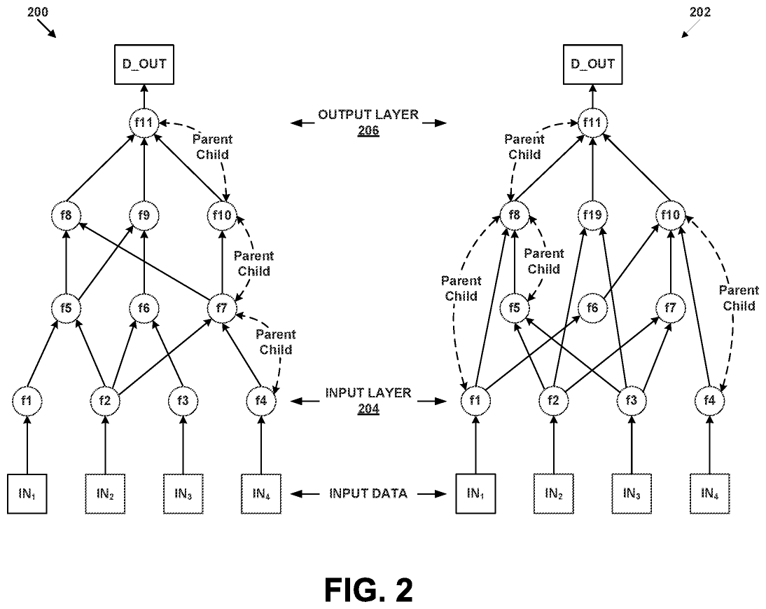

The neurons and layers of feed-forward neural networks may be arranged in tree-like structures. FIG. 2 is a conceptual illustration of two types of tree-like artificial neural network. In particular, ANN 200 depicts a feed-forward neural network having strict tree-like structure, while ANN 200 depicts a feed-forward neural network having non-strict tree-like. Both ANNs have an input layer 204 having four neurons f1, f2, f3, and f4, and an output layer 206 having a single neuron f11. Neurons are also referred to as “nodes” in describing their configuration and connections in a neural network. The four input neurons in the example are referred to as “leaf-nodes,” and the single output neuron is referred to as a “root node.”

In each ANN, neurons f5, f6, and f7 reside in a first “hidden layer” after the input layer 204, and neurons f8, f9, and f10 reside in a second hidden layer, which is also just before the output layer 206. The neurons in the hidden layers are also referred to as “hidden node” and/or “non-leaf nodes.” Note that the root node is also a non-leaf node. In addition, there could be ANNs having more than two hidden layers, or even just one hidden layer.

Input data IN₁, IN₂, IN₃, and IN₄ are input to the input neurons of each layer, and a single output D_OUT is output from the output neuron of each ANN. Connections between neurons (directed arrows in FIG. 2) correspond to activations fed forward from one neuron to the next. In particular, one or more nodes that provide input to a given node are referred to as “child nodes” of the given node, and the given node is referred to as the “parent node” of the child nodes. For the purposes of the discussion herein, strict tree-like ANNs, such as ANN 200, are distinguished from non-strict tree-like ANNs, such as ANN 202, by the types of parent-child connections they each have.

More specifically, in a strict tree-like ANN, the each child node of a parent node resides in a layer immediately prior to the layer in which the parent node resides. Three examples are indicated in ANN 200. Namely, f4 which is a child of f7 resides in the layer immediately prior to the f7's layer. Similarly, f7 which is a child of f10 resides in the layer immediately prior to the f10's layer, and f10 which is a child of f11 resides in the layer immediately prior to the f11's layer. It may be seen by inspection that the same relationship holds for all the connected nodes of ANN 200.

In a non-strict tree-like ANN, the each child node of a parent node resides in a layer prior to the layer in which the parent node resides, but it need not be the immediately prior layer. Three examples are indicated in ANN 202. Namely, f1 which is a child of f8 resides two layers ahead of f8's layer. Similarly, f4 which is a child of f10 resides two layers ahead of f10's layer. However, and f5 which is a child of f8 resides in the layer immediately prior to the f8's layer. Thus, a non-strict tree-like ANN may include a mix of inter-layer relationships.

Feed-forward neural networks (especially “deep,” i.e., ones with many layers) have been demonstrated to be quite successful in their predicative capabilities due in part to their ability to implicitly decompose complex objects into their constituent parts. This may be particularly the case for “convolutional” neural networks (CNNs), commonly used in computer vision. In CNNs, the weights in each layer are tied together, which tends to force the neurons to learn increasingly complex visual features, from simple edge detectors all the way to complex shapes such as human eyes, mouths, faces, and so on.

There has been recent interest in extending neural networks to learning from structured objects, such as graphs. A range of architectures have been proposed for this purpose, many of them based on various generalizations of the notion of convolution to these domains.

One particular architecture, which makes the part-based aspect of neural modeling very explicit, is that of “compositional networks” (“comp-nets”), introduced previously by the inventor. In accordance with example embodiments, comp-nets may represent a structured object X in terms of a decomposition of X into a hierarchy of parts, subparts, subsubparts, and so on, down to some number of elementary parts {eᵢ}. Referring to the parts, subparts, subsubparts, and so on, simply as “parts” or “subsystems” Pᵢ, the decomposition may be considered as forming a so-called “composition scheme” of a collection of P that make up the hierarchy.

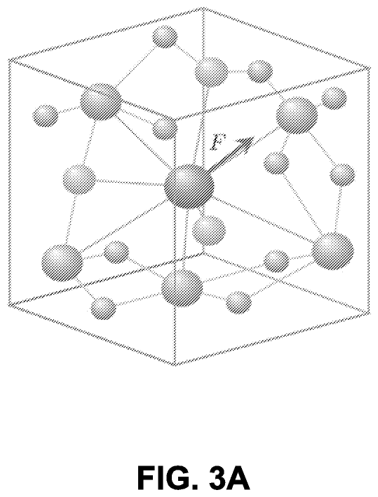

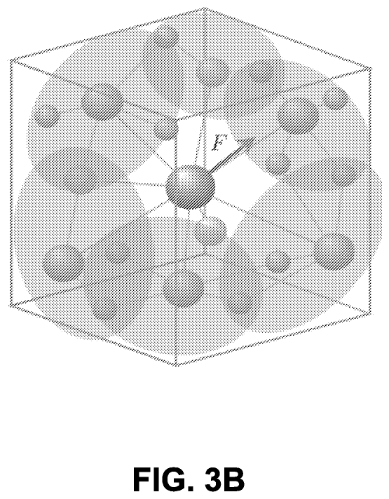

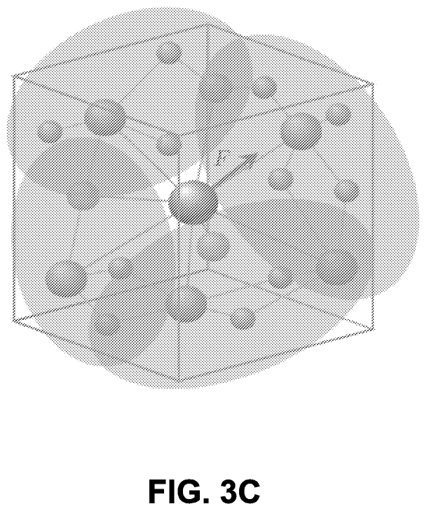

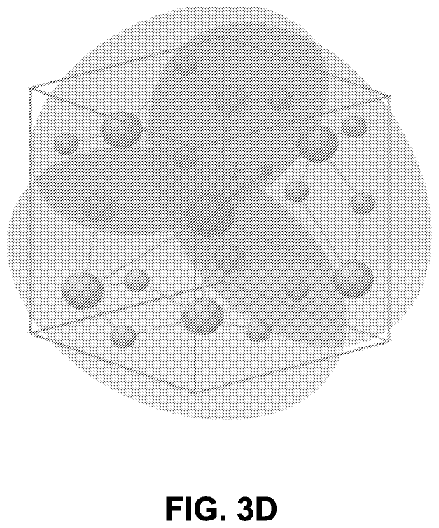

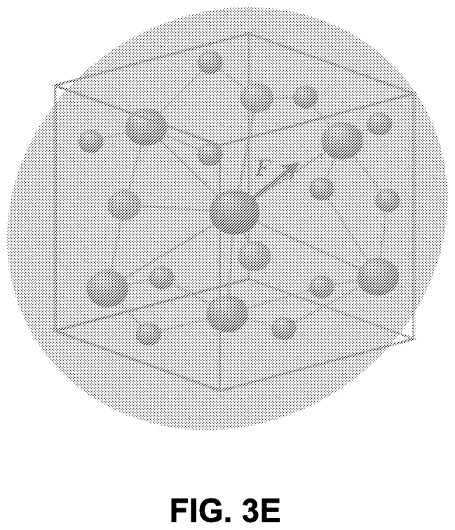

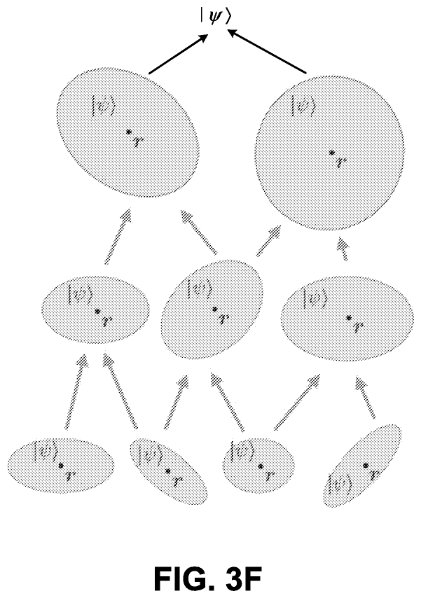

FIGS. 3A-3F illustrate conceptually the decomposition of an N-body physical system, such as a molecule, into an example hierarchy of subsystems. FIG. 3A first shows the example N-body physical system made up of constituent particles, such as atoms of a molecule. In the figure, and arrow point from a central particle and labeled F may represent the aggregate or total vector force on the central particle due to the physical interaction of the other particles. These might be electrostatic or other inter-atomic forces, for example.

By way of example, FIG. 3B shows a first level of the subsystem hierarchy. In the illustration, particular groupings of the particles represent a first level of subsystem or subparts. As shown, there appears to be six grouping, corresponding to six subparts. FIG. 3C show a next (second) level of the subsystem hierarchy, again by way of example. In this case, there are four groupings, corresponding to four subparts. Similarly, FIG. 3D shows the third level of groupings, this one having three subsystems, and FIG. 3E shows the top level of the hierarchy, having a single grouping that includes all of the lower level subsystems. In practice, the decomposition may be determined according to known or expected properties of the physical system under consideration. It will be appreciated, however, that the conceptual illustrations of FIGS. 3A-3E do not necessarily convey any such physical considerations. Rather, they merely depict the decomposition concept for the purposes of the discussion herein.

FIG. 3F illustrates how a decomposition scheme may be translated to a comp-net architecture. In accordance with example embodiments, each subsystem of a hierarchy may correspond to a node (or neuron) of an ANN in which successive layers represent successive layers of the compositional hierarchy. In accordance with at least some example embodiments, and as described in detail below, each subsystem may be described by a spatial position vector and an internal state vector. This is indicated by the labels r and |ψ> in each of the subsystems shown in FIG. 3F. In the comp-net representation, the internal state vector of each subsystem may be computed as the activation of the corresponding neuron. The inputs to each non-leaf node may be the activation of one or more child nodes of one or more prior layers, each child node representing a subsystem of a lower level of the hierarchy.

Returning to consideration of the decomposition and the composition scheme, since each part Pᵢ can be a sub-part of more than one higher level part, the composition scheme is not necessarily a strict tree, but is rather a DAG (directed acyclic graph). An exact definition, in accordance with example embodiments, is as follows.

Definition 1. Let X be a compound object with n elementary parts ε={e₁, . . . , eₙ}. A “composition scheme” D for X is a directed acyclic graph (DAG) in which each node nᵢ is associated with some subset Pᵢ of ε (these subsets are called the parts of X) in such a way that

1. If nᵢ is a leaf node, then Pᵢ contains a single elementary part e_{ξ(i)}.

2. D has a unique root node nᵢ, which corresponds to the entire set {e₁ . . . , eₙ}.

3. For any two nodes nᵢ and nⱼ, if nᵢ is a descendant of nⱼ, then Pᵢ⊂Pⱼ.

In accordance with example embodiments, a comp-net is a composition scheme that may be reinterpreted as a feed-forward neural network. In particular, in a comp-net each neuron nᵢ also has an activation fᵢ. For leaf nodes, fᵢ may be some simple pre-defined vector representation of the corresponding elementary part e(i). For internal nodes, fᵢ may be computed from the activations f_{ch}_{₁}, . . . , f_{ch}_{ₖ} of the children of nᵢ by the use of some aggregation function Φ(f_{ch}_{₁}, . . . , f_{ch}_{ₖ}) similar to equation (1). Finally, the output of the comp-net is the output of the root node nᵣ.

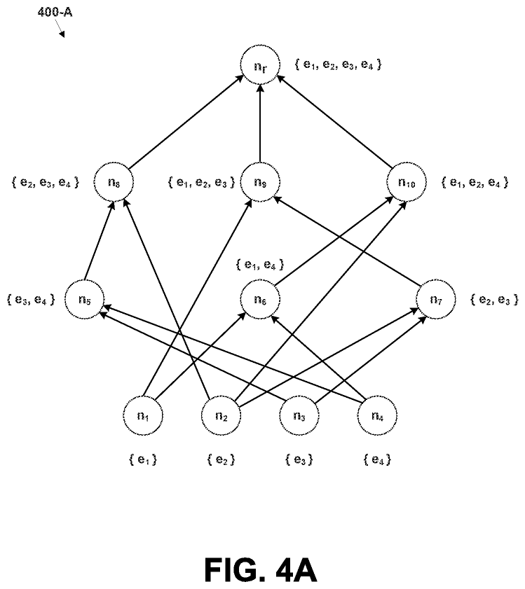

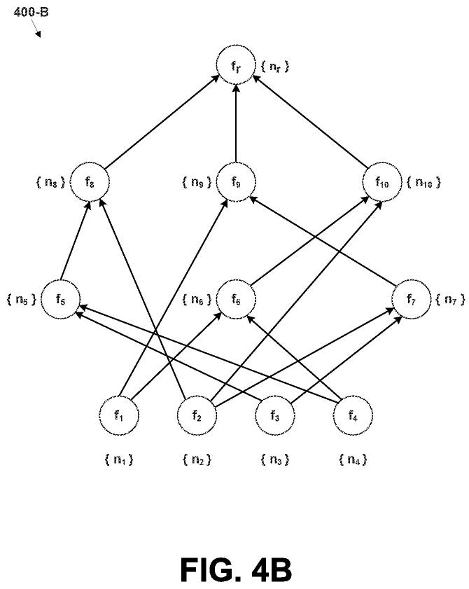

FIGS. 4A and 4B further illustrate by way of example a translation from a hierarchical composition scheme of a compound object to a corresponding compositional neural network (comp-net). Specifically, FIG. 4A depicts a composition scheme 400-A in which leaf-nodes n₁, n₂, n₃, and n₄ of the first (lowest) level of the hierarchy correspond to single-element subsystems {e₁}, {e₂}, {e₃}, and {e₄}, respectively.

At the next (second) level up in the hierarchy, non-leaf nodes n₅, n₆, and n₇ each contain two-element subsystems, each subsystem being “built” from a respective combination of two first-level subsystems. For example, as shown, n₅ contains {e₃, e₄} from nodes n₃ and n₄, respectively. The arrows pointing from n₃ and n₄ to n₅ indicate this relationship.

At the third level up, non-leaf nodes n₈, n₉, and n₁₀ each contain three-element subsystems, each subsystem being built from a respective combination of subsystems from the previous levels. For example, as shown, n₁₀ contains {e₁, e₄} from the two-element subsystem at n₆, and {e₂} from the single-element subsystem at n₂. The arrows pointing from n₆ and n₂ to n₁₀ indicate this relationship.

Finally, at the top level, the (non-leaf) root node nᵢ contains all four elementary parts in subsystem {eᵢ, e₂, e₃, e₄} from the previous level. Note that subsystems at a given level above the lowest (“leaf”) level may overlap in terms of common (shared) elementary parts and/or common (shared) lower-level subsystems. It may also be seen by inspection that the example composition scheme 400-A corresponds to a non-strict tree-like structure.

FIG. 4B illustrates an example comp-net 400-B that corresponds to the composition scheme 400-A of FIG. 4A. In this illustration, the neurons {n₁}, {n₂}, . . . , {nᵣ} of comp-net 400-B correspond, respectively, to the nodes n₁, n₂, . . . , nᵣ of the composition scheme 400-A, and the arrow connecting the neurons correspond to the relationships between the nodes in the composition scheme. The neuron are also associated with respective activations f₁, f₂, . . . , fᵣ, as shown. Thus, as illustrated in this example, the activations f₃ and f₄ are inputs to neuron (node) {n₅}, which uses them in computing its activation f₅.

The inventor has previously detailed the behavior of comp-nets under transformations of X, in particular, how to ensure that the output of the network is invariant with respect to spurious permutations of the elementary parts, whilst retaining as much information about the combinatorial structure of X as possible. This is significant in graph learning, where X is a graph, e₁, . . . , eₙ are its vertices, and {Pᵢ} are subgraphs of different radii. The proposed solution, “covariant compositional networks” (CCNs), involves turning the {fᵢ} activations into tensors that transform in prescribed ways with respect to permutations of the elementary parts making up each Pᵢ.

Referring again to FIG. 3F, the activations of the nodes of a comp-net may describe states of the subsystems corresponding to the nodes. In a representation of a physical N-body system, the computation of the state of a given node may characterize physical interactions between the constituent subsystems of the given node. In accordance with example embodiments, and as described in detail below, the activations are constructed to ensure that the states are tensorial objects with spatial character, and in particular that they are covariant with rotations in the sense that they transform under rotations according to specific irreducible representations of the rotation group.

### IV. ANALYTICAL DESCRIPTION OF, AND THEORETICAL BASES FOR, COVARIANT COMP-NETS

A. Compositional Models for Atomic Environments

Decomposing complex systems into a hierarchy of interacting subsystems at different scales is a recurring theme in physics, from coarse graining approaches to renormalization group theory. The same approach applied to the atomic neighborhood lends itself naturally to learning force fields. For example, to calculate the aggregate force on the central atom, in a first approximation one might just sum up independent contributions from each of its neighbors. In a second approximation, one would also consider the modifying effect of the local neighborhoods of the neighbors. A third order approximation would involve considering the neighborhoods of the atoms in these neighborhoods, and so on.

The inventor has recognized that the compositional networks formalism is thus a natural framework for force field learning. In particular, comp-nets may be considered in which the elementary parts correspond to actual physical atoms, the internal nodes correspond to subsystems Pᵢ made up of multiple atoms. In accordance with example embodiments, the corresponding activation, now denoted ψᵢ, and referred to herein as the state of Pᵢ, may effectively be considered a learned coarse grained representation of Pᵢ. What makes physical problems different from, such as learning graphs, however, is their spatial character. In particular:
1. Each subsystem Pᵢ may now also be associated with a vector rᵢ∈ [symbol] ³ specifying its spatial position.
2. The interaction between two subsystems Pᵢ and Pⱼ depends not only on their relative positions, but also on their relative orientations. Therefore, ψᵢ and ψⱼ must also have spatial character, somewhat similarly to the terms of the monopole, dipole, quadrupole, etc. expansions, for example.
   If the entire the atomic environment is rotated around the central atom by some rotation R∈SO(3)³, the position vectors transform as rᵢ [symbol] Rrᵢ. Mathematically, the second point above says that the ψᵢ activations (states) must also transform under rotations in a predictable way, which is expressed by saying that they must be rotationally covariant.

Group Representations and N-Body Networks

Just as covariance to permutations is a critical constraint on the graph CCNs, covariance to rotations is the guiding principle behind CCNs for learning atomic force fields. To describe this concept in its general form, a starting assumption may be taken to be that any given activation ψ is representable as a d dimensional (complex valued) vector, and that the transformation that ψ undergoes under a rotation R is linear, i.e., ψ [symbol] ρ(R)ψ for some matrix ρ(R).

The linearity assumption is sufficient to guarantee that for R, R′∈SO(3), ρ(R)ρ(R′)=ρ(RR′). Complex matrix valued functions satisfying this criterion are called representations of the group SO(3). Standard theorems in representation theory indicate that any compact group G (such as SO(3)) has a sequence of so-called inequivalent irreducible representations ρ₀, ρ₁, . . . (“irreps,” for short), and that any other representation μ of G can be reduced into a direct sum of irreps in the sense that there is some invertible matrix C and sequence of integers τ₀, τ₁, . . . such that

μ(R)=C⁻¹[ [symbol] (R)]C. (2)

Here [symbol] is called the multiplicity of [symbol] in μ, and r=τ(τ₀, τ₁, . . . ) is called the type of μ. Another feature of the representation theory of compact groups is that the irreps can always be chosen to be unitary, i.e., ρ(R⁻¹)=ρ(R)⁻¹=ρ(R)^{†}, where M^{†} denotes the Hermitian conjugate (conjugate transpose) of the matrix M. In the following it may be assumed that irreps satisfy this condition. If μ is also unitary, then the transformation matrix C will be unitary too, so C⁻¹ may be replaced with C^{†}.

In the specific case of the rotation group SO(3), the irreps are sometimes called Wigner D-matrices. The [symbol] =0 irrep consists of the one dimensional constant matrices ρ₀(R)=(1), the [symbol] =0 irrep (up to conjugation) is equivalent to the rotation matrices themselves, while for general [symbol] , assuming that (θ, ϕ, ψ) are the Euler angles of R, [ [symbol] (R)]_{m,m′}=e^{iψm′} [symbol] (θ, ϕ), where [symbol] are the well known spherical harmonic functions. In general, the dimensionality of [symbol] is [symbol] +1, i.e., [symbol] (R)∈ [symbol] .

Definition 2. ψ∈ [symbol] ^{d} is said to be an SO(3)-covariant vector of type τ=(τ₀, τ₁, τ2, . . . ) if under the action of rotations it transforms as

ψ [symbol] [ [symbol] (R)]ψ. (3)

Setting

ψ= [symbol] (4)

[symbol]

∈ [symbol] may be called the (l, m)-fragment of ψ, and

[symbol]

=

[symbol]

may be called the [symbol] 'th part of ψ. A covariant vector of type τ=(0, 0, . . . , 0, 1), where the single 1 corresponds to τₖ, may be called an irreducible vector of order k or an irreducible ρₖ-vector. Note that a first order irreducible vector is just a scalar.

A benefit of the above definition is that each fragment [symbol] transforms in the very simple way [symbol] [symbol] [symbol] (R) [symbol] . Note that the terms “fragment” and “part” are not necessarily standard in the literature, but are used here for being useful in describing covariant neural architectures. Also note that unlike equation (2), there is no matrix C in equations (3) and (4). This is because if a given vector w transforms according to a general representation μ whose decomposition does include a nontrivial C, this matrix may be easily be factored out by redefining ψ as Cψ. Here [symbol] is sometimes also called the projection of ψ to the [symbol] 'th isotypic subspace of the representation space that ψ lives in, and ψ=ψ⁰⊕ψ¹⊕ . . . is called the isotypic decomposition of ψ. With these representation theoretic tools in hand, the concept of SO(3)-covariant N-body neural networks may be defined as follows.

Definition 3. Let S be a physical system made up of n particles ξ₁, . . . , ξₙ. An SO(3)-covariant N-body neural network N for S is a composition scheme D in which
1. Each node nⱼ, which may also be referred to as a “gate,” is associated with
   (a) a physical subsystem Pⱼ of S;
   (b) a vector rⱼ∈ [symbol] ³ describing the spatial position of Pⱼ;
   (c) a vector ψⱼ that that describes the internal state of Pⱼ and is type τⱼ covariant to rotations.
2. If nⱼ is a leaf node, then ψⱼ is determined by the corresponding particle ξ_{J}.
3. If nⱼ is a non-leaf node and its children are n_{ch}_{₁}, . . . , n_{ch}_{ₖ}, then ψⱼ is computed as

ψⱼ=Φⱼ(\hat{r}_{ch}_{₁}, . . . ,\hat{r}_{ch}_{ₖ},\hat{r}_{ch}_{₁}, . . . ,\hat{r}_{ch}_{ₖ},ψ_{ch}_{₁}, . . . ,ψ_{ch}_{ₖ}), (5)
where \hat{r}_{ch}_{₁}=r_{ch}_{ᵢ}−rⱼ and \hat{r}ᵢ=|\hat{r}ᵢ|. In the discussion herein, ϕⱼ is referred to as the local “aggregation rule.”
4. D has a unique root nᵣ, and the output of the network, i.e., the learned state of the entire system is ψᵣ. In the case of learning scalar valued functions, such as the atomic potential, ψᵣ is just a scalar.

In accordance with example embodiments, Definition 3 may be considered as defining a general architecture for learning the state of N-body physical systems with much wider applicability than just learning atomic potentials. Also in accordance with example embodiments the Φⱼ aggregation rules may be defined in such a way as to guarantee that each y is SO(3)-covariant. This is what is addressed in the following section.

B. Covariant Aggregation Rules

To define the aggregation function Φ to be used in SO(3)-covariant comp-nets, it may only be assumed that Φ is a polynomial in the relative positions \hat{r}_{ch}_{₁}, . . . , \hat{r}_{ch}_{ₖ}, the constituent state vectors ψ_{ch}_{₁} . . . , ψ_{ch}_{ₖ} and the inverse distances 1/\hat{r}_{ch}_{₁}, . . . , 1/\hat{r}_{ch}_{ₖ}. Specifically, it may be said that Φ is a (P,Q,S)-order aggregation function if each component of ψ=Φⱼ(\hat{r}_{ch}_{₁}, . . . , \hat{r}_{ch}_{ₖ}, \hat{r}_{ch}_{₁}, . . . , \hat{r}_{ch}_{ₖ}, ψ_{ch}_{₁} . . . , ψ_{ch}_{ₖ}) is a polynomial of order at most p in each component of r_{ch}_{ᵢ}, a polynomial of at most q in each component of ψ_{ch}_{ᵢ}, and a polynomial of order at most s in each 1/(\hat{r}_{ch}_{ᵢ}). Any such Φ can be expressed as

$$Φ ( … ) = ( {⊕}_{p , q , s} {r}_{{ch}_{1}}^{{⊗}_{{p}_{1}}} ⊗ … ⊗ {r}_{{ch}_{k}}^{{⊗}_{{p}_{k}}} ⊗ {ψ}_{{ch}_{1}}^{{⊗}_{{q}_{1}}} ⊗ … ⊗ {ψ}_{{ch}_{k}}^{{⊗}_{{q}_{k}}} \cdot {\hat{r}}_{{ch}_{1}}^{- {s}_{1}} \cdot … \cdot {\hat{r}}_{{ch}_{k}}^{- {s}_{k}} ) , \qquad ( 6 )$$

where p, q and s are multi-indices of positive integers with pᵢ≤P, qᵢ≤Q and sᵢ≤S, and [symbol] is a linear function. The tensor products appearing in equation (6) are formidably large object and in most cases may be impractical to compute explicitly. Accordingly, this equation is meant to emphasize that any learnable parameters of the network must be implicit in the linear operator [symbol] .

The more stringent requirements on [symbol] arise from the covariance criterion. The inventor has recognized that understanding these may be aided by the observation that for any sequence ρ₁, . . . , ρₚ of (not necessarily irreducible) representations of a compact group G, their tensor product

ρ(R)=ρ₁(R)⊗ρ₂(R)⊗ . . . ⊗ρₚ(R)

is also a representation of G. Consequently, ρ has a decomposition into irreps, similar to equation (2). As an immediate corollary, any product of SO(3) covariant vectors can be similarly decomposed. In particular, by applying the appropriate unitary matrix C, the sum of tensor products appearing in equation (6) can be decomposed into a sum of irreducible fragments in the form

$${⊕}_{𝓁 = 0}^{ℒ} {⊕}_{m = 1}^{{τ′}_{𝓁}} {φ}_{m}^{𝓁} = C ( {⊕}_{p , q , s} {r}_{{ch}_{1}}^{{⊗}_{{p}_{1}}} ⊗ … ⊗ {r}_{{ch}_{k}}^{{⊗}_{{p}_{k}}} ⊗ {ψ}_{{ch}_{1}}^{{⊗}_{{q}_{1}}} ⊗ … ⊗ {ψ}_{{ch}_{k}}^{{⊗}_{{q}_{k}}} \cdot {\hat{r}}_{{ch}_{1}}^{- {s}_{1}} \cdot … \cdot {\hat{r}}_{{ch}_{k}}^{- {s}_{k}} ) .$$

More explicitly,

$${φ}_{m}^{𝓁} = {T}_{m}^{𝓁} ( {⊕}_{p , q , s} {r}_{{ch}_{1}}^{{⊗}_{{p}_{1}}} ⊗ … ⊗ {r}_{{ch}_{k}}^{{⊗}_{{p}_{k}}} ⊗ {ψ}_{{ch}_{1}}^{{⊗}_{{q}_{1}}} ⊗ … ⊗ {ψ}_{{ch}_{k}}^{{⊗}_{{q}_{k}}} \cdot {\hat{r}}_{{ch}_{1}}^{- {s}_{1}} \cdot … \cdot {\hat{r}}_{{ch}_{k}}^{- {s}_{k}} ) , \qquad ( 7 )$$

where T₁⁰, . . . , T_{τ}_{₀}⁰, T₁¹, . . . , T_{τ}_{₂}⁰, . . . , T_{τ}_{_{L}}^{L} is an appropriate sequence of projection operators. In accordance with example embodiments, the following proposition may provide a foundational result.

Proposition 1. The output of the aggregation function of equation (6) is a τ-covariant vector if and only if [symbol] is of the form

[symbol]

( . . . )= [symbol] [symbol] [symbol] ₘ [symbol] _{′}. (8)

Equivalently, collecting all [symbol] fragments with the same [symbol] into a matrix [symbol] ∈ [symbol] , all ( [symbol] _{′,m})_{m′,m} weights into a matrix [symbol] ∈ [symbol] and reinterpreting the output of [symbol] as a collection of matrices rather than a single long vector, equation (8) may be expressed as

[symbol]

( . . . )=(\tilde{F}⁰W⁰,\tilde{F}¹W¹, . . . ,\tilde{F}^{L}W^{L}). (9)

Proposition 1 indicates that [symbol] is only allowed to mix [symbol] fragments with the same [symbol] , and that fragments can only be mixed in their entirety, rather than picking out their individual components. These are fundamental consequences of equivariance. However, there are no further restrictions on the ( [symbol] mixing matrices.

In accordance with example embodiments, in an N-body neural network, the [symbol] matrices are shared across (some subsets of) nodes, and it is these mixing (weight) matrices that the network learns from training data. The [symbol] matrices can be regarded as generalized matrix valued activations. Since each [symbol] interacts with the [symbol] matrices linearly, the network can be trained the usual way by backpropagating gradients of whatever loss function is applied to the output node nᵣ, whose activation may typically be scalar valued.

It may be noted that N-body neural networks have no additional nonlinearity outside of Φ, since that would break covariance. In contrast, in most existing neural network architectures, as explained above, each neuron first takes a linear combination of its inputs weighted by learned weights and then applies a fixed pointwise nonlinearity, a. In accordance with architecture of N-body neural networks as described by way of example herein, the nonlinearity is hidden in the way that the [symbol] fragments are computed, since a tensor product is a nonlinear function of its factors. On the other hand, mixing the resulting fragments with the [symbol] weight matrices is a linear operation. Thus, in N-body neural networks as describe herein, the nonlinear part of the operation precedes the linear part.

The generic polynomial aggregation function of equation (6) may be too general to be used in a practical N-body network, and may be too costly computationally. Instead, in accordance with example embodiments, a few specific types of low order gates may be used, such as those described below

Zeroth Order Interaction Gates

Zeroth order interaction gates aggregate the states of their children and combine them with their relative position vectors, but do not capture interactions between the children. A simple example of such a gate would be one where

Φ( . . . )= [symbol] (Σᵢ₌₁^{k}(ψ_{ch}_{ᵢ}⊗\hat{r}_{ch}_{ᵢ}),Σᵢ₌₁^{k}\hat{r}_{ch}_{ᵢ}⁻¹(ψ_{ch}_{ᵢ}⊗\hat{r}_{ch}_{ᵢ}),Σᵢ₌₁^{k}\hat{r}_{ch}_{ᵢ}(ψ_{ch}_{ᵢ}⊗\hat{r}_{ch}_{ᵢ})). (10)

Note that the summations in these formulae ensure that the output is invariant with respect to permuting the children and also reduce the generality of equation (6) because the direct sum is replaced by an explicit summation (this can also be interpreted as tying some of the mixing weights together in a particular way). Let L be the largest [symbol] for which [symbol] ≠0 in the inputs. In the L=0 case each ψ_{ch}_{ᵢ} state is a scalar quantity, such as electric charge. In the L=1 case it is a vector, such as the dipole moment. In the L=2 case it can encode the quadropole moment, and so on. A gate of the above form can learn how to combine such moments into a single (higher order) moment corresponding to the parent system.

It may be instructive to see how many parameters a gate of this type has. For this purpose, the simple case that each ψ_{ch}_{ᵢ} is of type r=(1, 1, . . . , 1) (up to [symbol] =L) may be assumed. The type of \hat{r}_{ch}_{ᵢ} is (0, 1). According to the Clebsch-Gordan rules, as described in more detail below, the product of two such vectors is a vector of type (1, 3, 2, . . . , 2, 1) (of length L+1). It may be further assume that desired output type is again τ=(1, 1, . . . , 1) of length L. This means that the [symbol] =L+1 fragment does not even have to be computed, and the size of the weight matrices appearing in equation (9) are

W₀∈ [symbol] ^{1×3} W₁∈ [symbol] ^{1×9} W₂∈ [symbol] ^{1×6} . . . W_{L}∈ [symbol] ^{1×6}.

The size of these matrices changes dramatically as more “channels” are allowed. For example, if each of the input states are of type τ=(c, c, . . . , c), the type of ψ_{ch}_{ᵢ}⊗\hat{r}_{ch}_{ᵢ} becomes (c, 3c, 2c, . . . , 2c, 1c). Assuming again an output of type τ=(c, c, . . . , c), the weight matrices become

[symbol]

W₀∈ [symbol] ^{c×3c} W₁∈ [symbol] ^{c×9c} W₂∈ [symbol] ^{c×6c} . . . W_{L}∈ [symbol] ^{c×6c}.

[symbol]

In many networks, however, the number of channels increases with height in the network. Allowing the output type to be as rich as possible, without inducing linear redundancies, the output type becomes (3c, 9c, 6c, . . . , 6c, 3c), and

W₀∈ [symbol] ^{3c×3c} W₁∈ [symbol] ^{9c×9c} W₂∈ [symbol] ^{9c×6c} . . . W_{L}∈ [symbol] ^{6c×6c}.

First Order Interaction Gates

In first order interaction, gates each of the children interact with each other, and the parent aggregates these pairwise interactions. A simple example would be computing the total energy of a collection of charged bodies, which might be done with a gate of the form

Φ( . . . )= [symbol] (Σ_{i,j=1}^{k}(ψ_{ch}_{ᵢ}⊗ψ_{ch}_{ⱼ}⊗\hat{r}_{ch}_{ᵢ}⊗\hat{r}_{ch}_{ⱼ}),Σ_{i,j=1}^{k}\hat{r}_{ch}_{ᵢ}⁻¹\hat{r}_{ch}_{ⱼ}¹(ψ_{ch}_{ᵢ}⊗ψ_{ch}_{ⱼ}⊗\hat{r}_{ch}_{ᵢ}⊗\hat{r}_{ch}_{ⱼ}),Σ_{i,j=1}^{k}\hat{r}_{ch}_{ᵢ}⁻²\hat{r}_{ch}_{ⱼ}²(ψ_{ch}_{ᵢ}⊗ψ_{ch}_{ⱼ}⊗\hat{r}_{ch}_{ᵢ}⊗\hat{r}_{ch}_{ⱼ}),Σ_{i,j=1}^{k}\hat{r}_{ch}_{ᵢ}⁻³\hat{r}_{ch}_{ⱼ}⁻³(ψ_{ch}_{ᵢ}⊗ψ_{ch}_{ⱼ}⊗\hat{r}_{ch}_{ᵢ}⊗\hat{r}_{ch}_{ⱼ})). (11)

Generalizing equation (6) slightly, if the interaction only depends on the relative positions of the child systems, another form that may be used is

Φ( . . . )= [symbol] (Σ_{i,j=1}^{k}(ψ_{ch}_{ᵢ}⊗ψ_{ch}_{ⱼ}⊗\hat{r}_{ch}_{ᵢ}_{,ch}_{ⱼ}),Σ_{i,j=1}^{k}\hat{r}_{ch}_{ᵢ}_{,ch}_{ⱼ}⁻¹(ψ_{ch}_{ᵢ}⊗ψ_{ch}_{ⱼ}⊗\hat{r}_{ch}_{ᵢ}_{ch}_{ⱼ}),Σ_{i,j=1}^{k}\hat{r}_{ch}_{ᵢ}_{,ch}_{ⱼ}⁻²(ψ_{ch}_{ᵢ}⊗ψ_{ch}_{ⱼ}⊗\hat{r}_{ch}_{ᵢ}_{,ch}_{ⱼ}),Σ_{i,j=1}^{k}\hat{r}_{ch}_{ᵢ}_{,ch}_{ⱼ}⁻³(ψ_{ch}_{ᵢ}⊗ψ_{ch}_{ⱼ}⊗\hat{r}_{ch}_{ᵢ}_{,ch}_{ⱼ})), (12)

where \hat{r}_{ch}_{ᵢ}_{,ch}_{ⱼ}=\hat{r}_{ch}_{ᵢ}−\hat{r}_{ch}_{ⱼ} and \hat{r}_{ch}_{ᵢ}_{,ch}_{ⱼ}=|\hat{r}_{ch}_{ᵢ}_{,ch}_{ⱼ}|.

It will be appreciated that in the above, electrostatics was used only as an example. In practice, there would typically be no need to learn electrostatic interactions because they are already described by classical physics. Rather, using the zeroth and first order interaction gates may be envisaged as constituents of a larger network for learning more complicated interactions with no simple closed form that nonetheless broadly follow similar scaling laws as classical interactions.

C. Clebsch-Gordan Transforms

It may now be explained how the [symbol] projection maps appearing in equation (7) are computed. This is significant because the nonlinearities in N-body neural network as described herein are the tensor products, and, in accordance with example embodiments, the architecture needs to incorporate the ability to reduce vectors into a direct sum of irreducibles again straight after the tensor product operation.

To this end the inventor has recognized that representation theory provides a clear prescription for how this operation is to be performed. For any compact group G, given two irreducible representations [symbol] and [symbol] , the decomposition of [symbol] ⊗ [symbol] into a direct sum of irreducibles

[symbol]

(R)⊗ [symbol] (R)= [symbol] [ [symbol] [symbol] (R)] [symbol] (13)

is called the Clebsch-Gordan transform. In the specific case of SO(3), the κ multiplicities take on the very simple form

$${κ}_{{𝓁}_{1} , {𝓁}_{2}} ( 𝓁 ) = \{ \begin{matrix}1 if \vert {𝓁}_{1} - {𝓁}_{2} \vert \leq 𝓁 \leq {𝓁}_{1} + {𝓁}_{2} \\ 0 otherwise ,\end{matrix}$$

and the elements of the [symbol] matrices can also be computed relatively easily via closed form [symbol] formulae.

It may be seen immediately that equation (13) prescribes how to reduce the product of covariant vectors into irreducible fragments. Assuming for example that ψ₁ is an irreducible [symbol] vector and ψ₂ is an irreducible [symbol] vector, ψ₁⊗ψ₂ decomposes into irreducible fragments in the form

[symbol]

ψ₁⊗ψ₂= [symbol] where [symbol] = [symbol] (ψ₁⊗ψ₂),

and [symbol] is the part of [symbol] matrix corresponding to the [symbol] 'th “block.” Thus, in this case the operator [symbol] just corresponds to multiplying the tensor product by [symbol] . By linearity, the above relationship also extends to non-irreducible vectors. If ψ₁ is of type τ₁ and ψ₂ is of type τ₂, then

ψ₁⊗ψ₂=

[symbol]

where

κ_{τ}_{₁}_{,τ}_{₂}( [symbol] )= [symbol] [symbol] · [symbol] · [symbol] [| [symbol] ₁− [symbol] ₂|≤ [symbol] ≤ [symbol] ₁+ [symbol] ₂],

and [symbol] [·] is the indicator function. Once again, the actual [symbol] fragments are computed by applying the appropriate [symbol] matrix to the appropriate combination of irreducible fragments of ψ₁ and ψ₂. It is also clear that by applying the Clebsch-Gordan decomposition recurisively, a tensor product of any order may be decomposed, for example,

ψ₁⊗ψ₂⊗ψ₃⊗ . . . ⊗ψₖ=((ψ₁⊗ψ₂)⊗ψ₃)⊗ . . . ⊗ψₖ.

In practical computations of such higher order products, optimizing the order of operations and reusing potential intermediate results may be used to minimize computational cost.

### V. EXAMPLE METHOD

Example methods may be implemented as machine language instructions stored one or another form of the computer-readable storage, and accessible by the one or more processors of a computing device and/or system, and that, when executed by the one or more processors cause the computing device and/or system to carry out the various operations and functions of the methods described herein. By way of example, storage for instructions may include a non-transitory computer readable medium. In example operation, the stored instructions may be made accessible to one or more processors of a computing device or system. Execution of the instructions by the one or more processors may then cause the computing device or system to carry various operations of the example method.

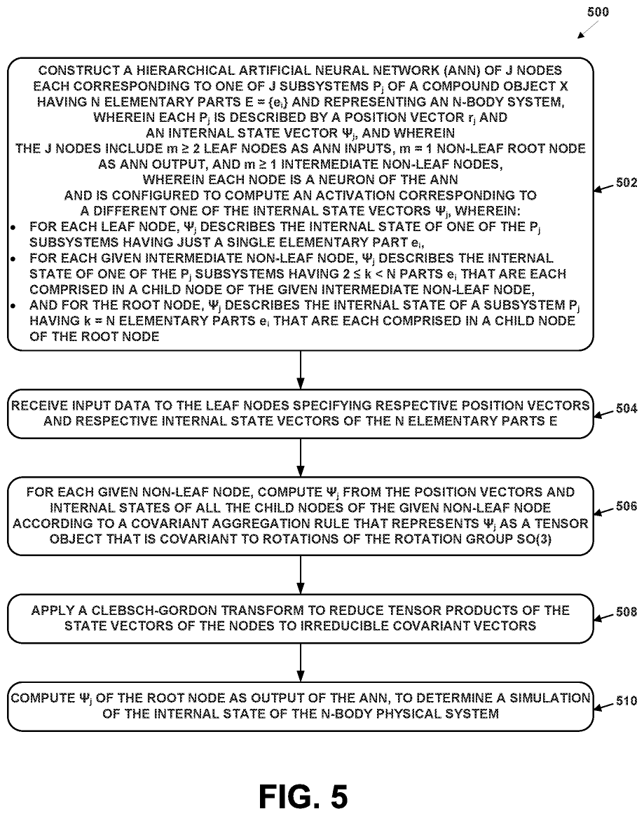

FIG. 5 is a flow chart of an example method 500, according to example embodiments. Specifically, example method 500 is a computational method for simulating an N-body physical system, wherein the N-body physical system is represented mathematically as a compound object X having N elementary parts E={eᵢ}, i=1, . . . , N, each eᵢ representing one of the N bodies of the N-body physical system. In accordance with example embodiments, X may be hierarchically decomposed into J subsystems, Pⱼ, j=1, . . . , J, each Pⱼ may include one or more of the elementary parts of E. Further, each Pⱼ may be described by a position vector rⱼ and an internal state vector ψⱼ. The steps of example method 500 may be carried out by a computing device, such as computing device 100.

At step 502, a hierarchical artificial neural network (ANN) having J nodes each corresponding to one of the J subsystems, may be constructed. In the context of a computer-implemented method, “constructing” an ANN may correspond to implementing the ANN in software or other machine language code. This may entail implementing data structures and operational and/or functional objects according to predefined classes as specified in various instructions, for example. The J nodes may m≥2 leaf nodes as ANN inputs, m=1 non-leaf root node as ANN output, and m≥1 intermediate non-leaf nodes. Each node may be considered a neuron of the ANN and may be configured to compute an activation corresponding to a different one of the internal state vectors ψⱼ according to node type. In particular, for each leaf node, ψⱼ may describe the internal state of a respective one of the Pⱼ subsystems having just a single elementary part eᵢ; for each given intermediate non-leaf node, ψⱼ may describe the internal state of a respective one of the Pⱼ subsystems having 2≤k<N parts eᵢ that are each comprised in a child node of the given intermediate non-leaf node; and for the root node, ψⱼ may describe the internal state of a subsystem Pⱼ having k=N elementary parts eᵢ that are each part of a child node of the root node.

At step 502, the computing device may receive input data to the leaf nodes specifying respective position vectors and respective internal state vectors of the N elementary parts E.

At step 506, for each given non-leaf node, ψⱼ may be computed from the position vectors and internal states of all the child nodes of the given non-leaf node according to a covariant aggregation rule that represents ψⱼ as a tensor object that is covariant to rotations of the rotation group SO(3).

At step 508, a Clebsch-Gordan transform may be applied to reduce tensor products of the state vectors of the nodes to irreducible covariant vectors.

Finally, at step 510, ψⱼ of the root node may be computed as output of the ANN. As such, the result may take the form of, or correspond to, a simulation of the internal state of the N-body physical system.

In accordance with example embodiments, the tensor products of the state vectors and application of the Clebsch-Gordan transform entail mathematical operations that are nonlinear. Further applying the Clebsch-Gordan transform to reduce the tensor products of the state vectors of the nodes to irreducible covariant vectors may entail applying the nonlinear operations in Fourier space.

In accordance with example embodiments, the m≥2 leaf nodes may form an input layer of the hierarchical ANN, the m=1 non-leaf root node may form an single-node output layer of the hierarchical ANN, and the m≥1 intermediate non-leaf nodes may be distributed among m≥1 intermediate layers of the hierarchical ANN. In addition, the hierarchical ANN is one of a strict tree-like structure, or a non-strict tree-like structure. As described above, in a strict tree-like structure, each successive layer after the input layer may include one or more parent nodes of one or more child nodes that reside only in an immediately preceding layer. As also described above, in a non-strict tree structure, each successive layer after the input layer may include one or more parent nodes of one or more child nodes that reside among more than preceding layer.

In further accordance with example embodiments, each given non-leaf node computing ψⱼ from the position vectors and internal states of all the child nodes of the given non-leaf node may entail the given non-leaf node receiving the activation of each of its child nodes. In an example embodiment, the activation of each given child node may correspond to the internal state of the given child node.

In accordance with example embodiments, the J subsystems may correspond to a hierarchy of substructures of the compound object X, from smallest to largest, the largest corresponding to the entirety of X. In this scheme, each of the Pⱼ subsystems that has just a single elementary part eᵢ may correspond to a single one of the smallest substructures, the subsystem Pⱼ that has k=N elementary parts eᵢ may correspond to the largest substructure, and wherein the Pⱼ subsystems that have 2≤k<N parts eᵢ may correspond to substructures between the smallest and largest.

In further accordance with example embodiments, the J subsystems may correspond to a hierarchy of substructures of the compound object X, such that each node of the hierarchical ANN corresponds to one of the substructures of the compound object X. As such, each respective non-leaf node may correspond to a respective substructure of the compound object X that includes the substructures of all of the child nodes of the respective non-leaf node, and each respective leaf node may correspond to a particular substructure of the compound object X comprising a single elementary part eᵢ. In an example embodiment, the internal state of each given subsystem may then correspond to a respective potential energy function due to physical interactions among the substructures of the child nodes of the node corresponding to the given subsystem.

In still further accordance with example embodiments, the hierarchical ANN may include adjustable weights shared among two or more of the nodes, such that the method further comprises training the ANN to learn the potential energy functions of all of the subsystems by adjusting the weights of the nodes corresponding to the subsystems.

In further accordance with example embodiments, training the ANN to learn the potential energy functions may entail providing training data to the input layer, where the training data includes for the N-body physical system one or more known training sets. Each training set may include (i) a given configuration of position vectors, and (ii) a known potential function for the given configuration. Training may thus entail, for each of the training sets, comparing a computed potential function output from the non-leaf root node with the known potential function for the given configuration, and based on the comparing, adjusting the weights to achieve agreement, to within a threshold level, between the computed potential functions and the known potential functions across the training sets.

Further, as the training sets may be associated with multiple different known configurations, an N-body comp-net may learn to recognize potentials from multiple examples. In this way, the N-body comp-net may later be applied to provide simulation results for new configurations that have not been previously analyzed. And as discussed above, learning molecular potentials represents a non-limiting example of physical properties or characteristics that an N-body comp-net may learn during training, and later predict from “live” testing data.

In further accordance with example embodiments, each of the training sets may include empirical measurements of the N-body physical system, ab initio computations of forces and energies of the N-body physical system, or a mixture of both.

In an example embodiment, method 500 may be applied to simulate molecules. As such, the compound object X may be or include molecules, and each elementary part eᵢ may be an atom. In this application of method 500, ψⱼ for each node may represent atomic potentials and forces experienced by each corresponding subsystem Pⱼ due the presence and relative positions of each of the other Pⱼ subsystems.

### CONCLUSION

Using neural networks to learn to the behavior and properties of complex physical systems shows considerable promise. However, physical systems have nontrivial invariance properties (in particular, invariance to translations, rotations and the exchange of identical elementary parts) that must be strictly respected.

Methods and systems disclosed here employ a new type of generalized convolutional neural network architecture, N-body networks, which provides a flexible framework for modeling interacting systems of various types, while taking into account these invariances (symmetries). An example application for N-body networks learning atomic potentials (force fields) for molecular dynamics simulations. However, N-body networks may be used more broadly, for modeling a variety of systems.

N-body networks are distinguished from earlier neural network models for physical systems in that
1. The model is based on a hierarchical (but not necessarily strictly tree-like) decomposition of the system into subsystems at different levels, which is directly reflected in the structure of the neural network.
2. Each subsystem is identified with a “neuron” (or “gate”) nᵢ in the network, and the output (activation) ψᵢ of the neuron becomes a representation of the subsystem's internal state.
3. The ψᵢ states are tensorial objects with spatial character, in particular they are covariant with rotations in the sense that they transform under rotations according to specific irreducible representations of the rotation group. The gates are specially constructed to ensure that this covariance property is preserved through the network.
4. Unlike most other neural network architectures, the nonlinearities in N-body networks are not pointwise operations, but are applied in “Fourier space,” i.e., directly to the irreducible parts of the state vector objects. This is only possible because (a) the nonlinearities arise as a consequence of taking tensor products of covariant objects (b) the tensor products are decomposed into irreducible parts by the Clebsch-Gordan transform.

Advantageously, the last of these ideas may be particularly promising, because it allows for constructing neural that operate entirely in Fourier space, and use tensor products combined with Clebsch-Gordan transforms to induce nonlinearities.

While example embodiments of N-body networks have been described in terms of molecular or atomic systems and potentials, applicability may be significantly broader. In particular, while ψⱼ of a given subsystem has be described as the “internal state” of a system (or subsystem), this should not be interpreted as limiting the scope with respect to other applications.

In addition, application of N-body networks to learning the energy function of the system is also just one possible non-limiting example. In particular, the architecture can also be used for learning a variety of other things, such as solubility, affinity for binding to some kind of target, and as well as other physical, chemical, or biological properties.

As a further example of broader applicability, DFT (e.g., ab initio) and other models that may provide training data and models for N-body networks can provide forces in addition to energies. The force information may be relatively easily integrated into the N-body network framework because the force is the gradient of the energy, and neural networks already propagate gradients. This opens the possibility of learning from derivatives as well.

More generally, neural networks may be flexibly extended and/or applied in joint operation. As such, the example application described herein may be considered a convenient supervised learning setting for illustrative purposes. However, applying the Clebsch-Gordan approach to N-body comp-nets may also be used (possibly as part of a larger architecture) to optimize the structure of atomic systems or generate new molecules for a particular goal, such as drug design.

Example embodiments herein provide a novel and efficient approach to computationally simulating an N-body physical system with covariant, compositional neural networks.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. A method for computationally simulating an N-body physical system, wherein the N-body physical system is represented mathematically as a compound object X having N elementary parts E={eᵢ}, i=1, . . . , N, each eᵢ representing one of the N bodies of the N-body physical system, wherein X is hierarchically decomposed into J subsystems, Pⱼ, j=1, . . . , J, each Pⱼ comprising one or more of the elementary parts of E, and wherein each Pⱼ is described by a position vector rⱼ and an internal state vector ψⱼ, the method being implemented on a computing device and comprising:
constructing a hierarchical artificial neural network (ANN) having J nodes each corresponding to one of the J subsystems, the J nodes including m≥2 leaf nodes as ANN inputs, m=1 non-leaf root node as ANN output, and m≥1 intermediate non-leaf nodes, wherein each node is a neuron of the ANN and is configured to compute an activation corresponding to a different one of the internal state vectors ψⱼ, and wherein:
for each leaf node, ψⱼ describes the internal state of a respective one of the Pⱼ subsystems having just a single elementary part eᵢ,
for each given intermediate non-leaf node, ψⱼ describes the internal state of a respective one of the Pⱼ subsystems having 2≤k<N parts eᵢ that are each comprised in a child node of the given intermediate non-leaf node,
and for the root node, ψⱼ describes the internal state of a subsystem Pⱼ having k=N elementary parts eᵢ that are each comprised in a child node of the root node;
at the computing device, receiving input data to the leaf nodes specifying respective position vectors and respective internal state vectors of the N elementary parts E;
for each given non-leaf node, computing ψⱼ from the position vectors and internal states of all the child nodes of the given non-leaf node according to a covariant aggregation rule that represents ψⱼ as a tensor object that is covariant to rotations of the rotation group SO(3);
applying a Clebsch-Gordan transform to reduce tensor products of the state vectors of the nodes to irreducible covariant vectors; and
computing ψⱼ of the root node as output of the ANN, to determine a simulation of the internal state of the N-body physical system.

2. The method of claim 1, wherein the tensor products of the state vectors and application of the Clebsch-Gordan transform comprise mathematical operations that are nonlinear,
and wherein applying the Clebsch-Gordan transform to reduce the tensor products of the state vectors of the nodes to irreducible covariant vectors comprises applying the nonlinear operations in Fourier space.

3. The method of claim 1, wherein the m>2 leaf nodes form an input layer of the hierarchical ANN, the m=1 non-leaf root node forms an single-node output layer of the hierarchical ANN, and the m≥1 intermediate non-leaf nodes are distributed among m≥1 intermediate layers of the hierarchical ANN,
and wherein the hierarchical ANN is one of:
a strict tree structure, each successive layer after the input layer comprising one or more parent nodes of one or more child nodes that reside only in an immediately preceding layer; or
a non-strict tree structure, each successive layer after the input layer comprising one or more parent nodes of one or more child nodes that reside among more than preceding layer.

4. The method of claim 3, wherein each given non-leaf node computing ψⱼ from the position vectors and internal states of all the child nodes of the given non-leaf node comprises the given non-leaf node receiving the activation of each of its child nodes, the activation of each given child node comprising the internal state of the given child node.

5. The method of claim 1, wherein the J subsystems correspond to a hierarchy of substructures of the compound object X, from smallest to largest, the largest corresponding to the entirety of X,
wherein each of the Pⱼ subsystems that has just a single elementary part eᵢ corresponds to a single one of the smallest substructures,
wherein the subsystem Pⱼ that has k=N elementary parts eᵢ corresponds to the largest substructure,
and wherein the Pⱼ subsystems that have 2≤k<N parts eᵢ correspond to substructures between the smallest and largest.

6. The method of claim 1, wherein the J subsystems correspond to a hierarchy of substructures of the compound object X, and wherein each node of the hierarchical ANN corresponds to one of the substructures of the compound object X,
wherein each respective non-leaf node corresponds to a respective substructure of the compound object X comprising the substructures of all of the child nodes of the respective non-leaf node,
wherein each respective leaf node corresponds to a particular substructure of the compound object X comprising a single elementary part eᵢ,
and wherein the internal state of each given subsystem corresponds to a respective potential energy function due to physical interactions among the substructures of the child nodes of the node corresponding to the given subsystem.

7. The method of claim 6, wherein the hierarchical ANN comprises adjustable weights shared among two or more of the nodes,
and wherein the method further comprises training the ANN to learn the potential energy functions of all of the subsystems by adjusting the weights of the nodes corresponding to the subsystems.

8. The method of claim 7, wherein training the ANN to learn the potential energy functions comprises:
providing training data to the input layer, the training data comprising for the N-body physical system one or more known training sets, each including: (i) a given configuration of position vectors, and (ii) a known potential function for the given configuration;
for each of the training sets, comparing a computed potential function output from the non-leaf root node with the known potential function for the given configuration; and
based on the comparing, adjusting the weights to achieve agreement, to within a threshold level, between the computed potential functions and the known potential functions across the training sets.

9. The method of claim 8, wherein each of the training sets is at least one of empirical measurements of the N-body physical system, or ab initio computations of forces and energies of the N-body physical system.

10. The method of claim 1, wherein the compound object X is comprised of molecules, wherein each elementary part eᵢ is an atom,
and wherein ψⱼ for each node represents atomic potentials and forces experienced by each corresponding subsystem Pⱼ due the presence and relative positions of each of the other Pⱼ subsystems.

11. A computing device configured for computationally simulating an N-body physical system, wherein the N-body physical system is represented mathematically as a compound object X having N elementary parts E={eᵢ}, i=1, . . . , N, each eᵢ representing one of the N bodies of the N-body physical system, wherein X is hierarchically decomposed into J subsystems, Pⱼ, j=1, . . . , J, each Pⱼ comprising one or more of the elementary parts of E, and wherein each Pⱼ is described by a position vector rⱼ and an internal state vector ψⱼ, the computing device comprising:
one or more processors; and
memory configured to store computer-executable instructions that, when executed by the one or more processors, cause the computing device to carry out computational operations including:
constructing a hierarchical artificial neural network (ANN) having J nodes each corresponding to one of the J subsystems, the J nodes including m≥2 leaf nodes as ANN inputs, m=1 non-leaf root node as ANN output, and m≥1 intermediate non-leaf nodes, wherein each node is a neuron of the ANN and is configured to compute an activation corresponding to a different one of the internal state vectors ψⱼ, and wherein:
for each leaf node, ψⱼ describes the internal state of a respective one of the Pⱼ subsystems having just a single elementary part eᵢ,
for each given intermediate non-leaf node, ψⱼ describes the internal state of a respective one of the Pⱼ subsystems having 2≤k<N parts eᵢ that are each comprised in a child node of the given intermediate non-leaf node,
and for the root node, ψⱼ describes the internal state of a subsystem Pⱼ having k=N elementary parts eᵢ that are each comprised in a child node of the root node;
receiving input data to the leaf nodes specifying respective position vectors and respective internal state vectors of the N elementary parts E;
for each given non-leaf node, computing ψⱼ from the position vectors and internal states of all the child nodes of the given non-leaf node according to a covariant aggregation rule that represents ψⱼ as a tensor object that is covariant to rotations of the rotation group SO(3);
applying a Clebsch-Gordan transform to reduce a tensor product of the state vectors of the nodes to irreducible covariant vectors; and
computing ψⱼ of the root node as output of the ANN to determine the internal state of the N-body physical system.

12. The computing device of claim 11, wherein the tensor products of the state vectors and application of the Clebsch-Gordan transform comprise mathematical operations that are nonlinear,
and wherein applying the Clebsch-Gordan transform to reduce the tensor products of the state vectors of the nodes to irreducible covariant vectors comprises applying the nonlinear operations in Fourier space.

13. The computing device of claim 11, wherein the m≥2 leaf nodes form an input layer of the hierarchical ANN, the m=1 non-leaf root node forms an single-node output layer of the hierarchical ANN, and the m≥1 intermediate non-leaf nodes are distributed among m≥1 intermediate layers of the hierarchical ANN,
wherein the hierarchical ANN is one of:
a strict tree structure, each successive layer after the input layer comprising one or more parent nodes of one or more child nodes that reside only in an immediately preceding layer; or
a non-strict tree structure, each successive layer after the input layer comprising one or more parent nodes of one or more child nodes that reside among more than preceding layer,
and wherein each given non-leaf node computing ψⱼ from the position vectors and internal states of all the child nodes of the given non-leaf node comprises the given non-leaf node receiving the activation of each of its child nodes, the activation of each given child node comprising the internal state of the given child node.

14. The computing device of claim 11, wherein the J subsystems correspond to a hierarchy of substructures of the compound object X, from smallest to largest, the largest corresponding to the entirety of X,
wherein each of the Pⱼ subsystems that has just a single elementary part eᵢ corresponds to a single one of the smallest substructures,
wherein the subsystem Pⱼ that has k=N elementary parts eᵢ corresponds to the largest substructure,
and wherein the Pⱼ subsystems that have 2≤k<N parts eᵢ correspond to substructures between the smallest and largest.

15. The computing device of claim 11, wherein the J subsystems correspond to a hierarchy of substructures of the compound object X, and wherein each node of the hierarchical ANN corresponds to one of the substructures of the compound object X,
wherein each respective non-leaf node corresponds to a respective substructure of the compound object X comprising the substructures of all of the child nodes of the respective non-leaf node,
wherein each respective leaf node corresponds to a particular substructure of the compound object X comprising a single elementary part eᵢ,
and wherein the internal state of each given subsystem corresponds to a respective potential energy function due to physical interactions among the substructures of the child nodes of the node corresponding to the given subsystem.

16. The computing device of claim 15, wherein the hierarchical ANN comprises adjustable weights shared among two or more of the nodes,
and wherein the computational operations further comprise training the ANN to learn the potential energy functions of all of the subsystems by adjusting the weights of the nodes corresponding to the subsystems.

17. The computing device of claim 16, wherein training the ANN to learn the potential energy functions comprises:
providing training data to the input layer, the training data comprising for the N-body physical system one or more known training sets, each including: (i) a given configuration of position vectors, and (ii) a known potential function for the given configuration;
for each of the training sets, comparing a computed potential function output from the non-leaf root node with the known potential function for the given configuration; and
based on the comparing, adjusting the weights to achieve agreement, to within a threshold level, between the computed potential functions and the known potential functions across the training sets.

18. The computing device of claim 17, wherein each of the training sets is at least one of empirical measurements of the N-body physical system, or ab initio computations of forces and energies of the N-body physical system

19. The computing device of claim 11, wherein the compound object X is comprised of molecules, wherein each elementary part eᵢ is an atom,
and wherein ψⱼ for each node represents atomic potentials and forces experienced by each corresponding subsystem Pⱼ due the presence and relative positions of each of the other Pⱼ subsystems.

20. An article of manufacture comprising a non-transitory computer readable media having computer-readable instructions stored thereon for computationally simulating an N-body physical system, wherein the N-body physical system is represented mathematically as a compound object X having N elementary parts E={eᵢ}, i=1, . . . , N, each eᵢ representing one of the N bodies of the N-body physical system, wherein X is hierarchically decomposed into J subsystems, Pⱼ, j=1, . . . , J, each Pⱼ comprising one or more of the elementary parts of E, and wherein each Pⱼ is described by a position vector rⱼ and an internal state vector ψⱼ, and wherein the instructions, when executed by one or more processors of a computing device, cause the computing device to carry out operations including:
constructing a hierarchical artificial neural network (ANN) having J nodes each corresponding to one of the J subsystems, the J nodes including m≥2 leaf nodes as ANN inputs, m=1 non-leaf root node as ANN output, and m≥1 intermediate non-leaf nodes, wherein each node is a neuron of the ANN and is configured to compute an activation corresponding to a different one of the internal state vectors ψⱼ, and wherein:
for each leaf node, ψⱼ describes the internal state of a respective one of the Pⱼ subsystems having just a single elementary part eᵢ,
for each given intermediate non-leaf node, ψⱼ describes the internal state of a respective one of the Pⱼ subsystems having 2≤k<N parts eᵢ that are each comprised in a child node of the given intermediate non-leaf node,
and for the root node, ψⱼ describes the internal state of a subsystem Pⱼ having k=N elementary parts eᵢ that are each comprised in a child node of the root node;
receiving input data to the leaf nodes specifying respective position vectors and respective internal state vectors of the N elementary parts E;
for each given non-leaf node, computing ψⱼ from the position vectors and internal states of all the child nodes of the given non-leaf node according to a covariant aggregation rule that represents ψⱼ as a tensor object that is covariant to rotations of the rotation group SO(3);
applying a Clebsch-Gordan transform to reduce a tensor product of the state vectors of the nodes to irreducible covariant vectors; and
computing ψⱼ of the root node as output of the ANN to determine the internal state of the N-body physical system.
